(19) United States
(12) Patent Application Publication
(10) Pub. No.: US 2024/0029858 A1
(43) Pub. Date: Jan. 25, 2024

(54) SYSTEMS AND METHODS FOR GENERATING AND EVALUATING A MEDICAL PROCEDURE

(71) Applicant: INTUITIVE SURGICAL OPERATIONS, INC., Sunnyvale, CA (US)

(72) Inventors: Prasad V. Upadrasta, San Jose, CA (US); John Ryan Steger, Los Gatos, CA (US)

(21) Appl. No.: 18/255,352

(22) PCT Filed: Nov. 30, 2021
(86) PCT No.: PCT/US2021/061106

§ 371 Date: May 31, 2023

Related U.S. Application Data
(60) Provisional application No. 63/120,191, filed on Dec. 1, 2020.

(51) Int. Cl. G16H 20/40; G16H 40/20; G16H 10/60

(52) U.S. Cl. CPC G16H 20/40; G16H 40/20; G16H 10/60

(57) ABSTRACT

A system may comprise a processor and a memory having computer readable instructions stored thereon. The computer readable instructions, when executed by the processor, may cause the system to generate a procedure plan for performing a procedure with a robot-assisted manipulator. The procedure plan may be based on a first plurality of procedure inputs. The system may also generate a performance metric from the implementation of the procedure, evaluate the implemented procedure based on the performance metric to generate procedure evaluation information, and store the procedure evaluation information. The system may also generate a second procedure plan based on the stored procedure evaluation information and a second plurality of procedure inputs.

### CROSS-REFERENCED APPLICATIONS

This application claims the benefit of U.S. Provisional Application 63/120,191 filed Dec. 1, 2020, which is incorporated by reference herein in its entirety.

This application incorporates by reference in their entireties U.S. Provisional Application No. 63/120,175, filed Dec. 1, 2020, titled “SYSTEMS AND METHODS FOR GENERATING VIRTUAL REALITY GUIDANCE” and U.S. Provisional Application No. 63/120,140, filed Dec. 1, 2020, titled “SYSTEMS AND METHODS FOR PLANNING A MEDICAL ENVIRONMENT.”

### FIELD

The present disclosure is directed to systems and methods for robot-assisted medical procedures and more specifically to developing a medical environment plan based on a mode of operation for a robot-assisted medical system.

### BACKGROUND

Planning tools for performing medical procedures with teleoperational robotic or robot-assisted systems are often generic and not adaptable to a particular surgeon, patient, or other parameters. Additionally, planning tools may be static and non-responsive to information that may improve patient outcomes. Systems and methods are needed to assist medical personnel by providing procedure planning tools that are adapted to a variety of parameters and that evaluate implemented procedures to identify areas for improved efficiency and patient outcomes.

### SUMMARY

The embodiments of the invention are best summarized by the claims that follow the description.

Consistent with some embodiments, a system may comprise a processor and a memory having computer readable instructions stored thereon. The computer readable instructions, when executed by the processor, may cause the system to generate a procedure plan for performing a procedure with a robot-assisted manipulator. The procedure plan may be based on a first plurality of procedure inputs. The system may also generate a performance metric from the implementation of the procedure, evaluate the implemented procedure based on the performance metric to generate procedure evaluation information, and store the procedure evaluation information. The system may also generate a second procedure plan based on the stored procedure evaluation information and a second plurality of procedure inputs.

In some embodiments, a system may comprise a processor and a memory having computer readable instructions stored thereon. The computer readable instructions, when executed by the processor, may cause the system to receive a procedure type for a planned procedure of a robot-assisted manipulator, receive a set of patient information for a patient undergoing the planned procedure, and generate an analysis of prior procedure data based on the received procedure type and the set of patient information. The system may also generate a set of set-up instructions for the planned procedure based on the analysis.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a flowchart illustrating a method for generating and evaluating a procedure plan according to some embodiments.

FIGS. 2A-2I illustrates a user interface for a procedure plan.

FIG. 3 is a flowchart illustrating a method for generating set-up instructions for a medical procedure.

FIG. 4 is a schematic illustration of a robot-assisted medical system according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

Procedure planning tools may assist in the efficient, safe, and effective use of robot-assisted systems in a medical environment. Adaptive procedure plans may respond to unique inputs for a particular medical procedure and may incorporate improvements and efficiencies identified in prior procedures. As described below, evaluations and analysis conducted on prior procedures may be used to generate procedure plans, including set-up instructions for a robot-assisted system in a medical environment.

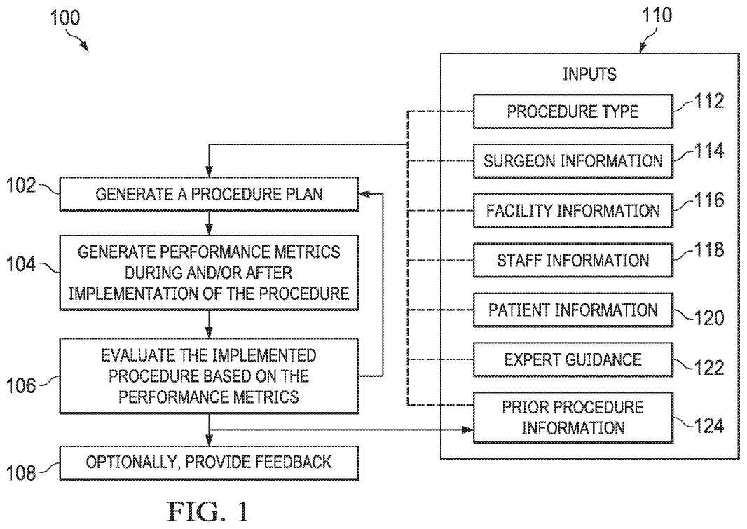

FIG. 1 is a flowchart illustrating a method 100 for generating and evaluating a procedure plan according to some embodiments. The methods described herein are illustrated as a set of operations or processes and are described with continuing reference to the additional figures. Not all of the illustrated processes may be performed in all embodiments of the methods. Additionally, one or more processes that are not expressly illustrated in may be included before, after, in between, or as part of the illustrated processes. In some embodiments, one or more of the processes may be implemented, at least in part, in the form of executable code stored on non-transitory, tangible, machine-readable media that when run by one or more processors (e.g., the processors of a control system) may cause the one or more processors to perform one or more of the processes. In one or more embodiments, the processes may be performed by a control system.

At a process 102, a procedure plan may be generated for using a robot-assisted medical system in a medical environment. The procedure plan may be developed based on various inputs 110 including, for example, a procedure type 112, surgeon information 114, facility information 116, staff information 118, patient information 120, expert guidance 122, and prior procedure information 124. In some embodiments, the inputs may be received at and the procedure plan may be displayed on, for example, a user interface device such as a display on an operator interface system, a stationary or movable auxiliary display in a medical environment, or a display on a mobile device such as a phone, tablet, camera, laptop or other portable device. In some embodiments, the user interface device may also measure, scan, image or otherwise record spatial information about the medical environment from within or proximate to the medical environment. Thus, the participants in the procedure, which may include, for example the surgeon, the clinical staff, and/or a mentor or supervisor, may review the procedure plan and make any adjustments to the plan prior to implementation of the plan.

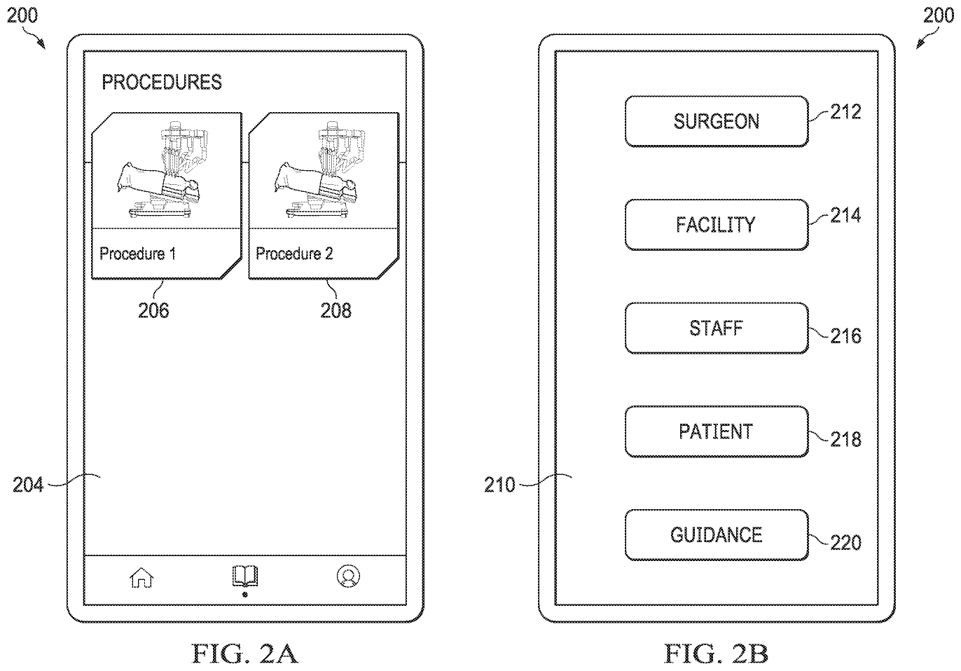

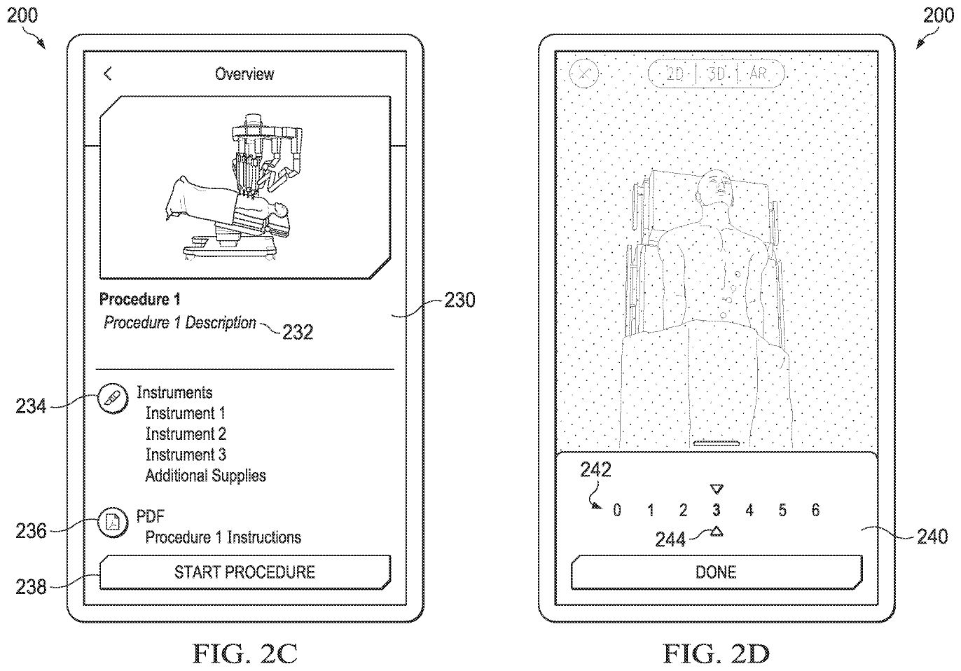

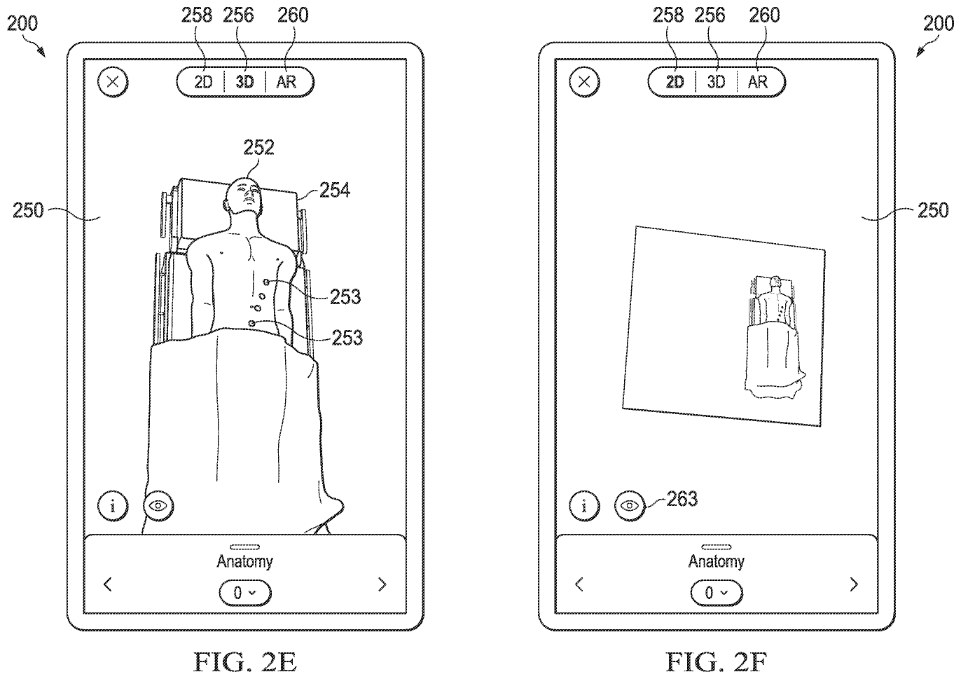

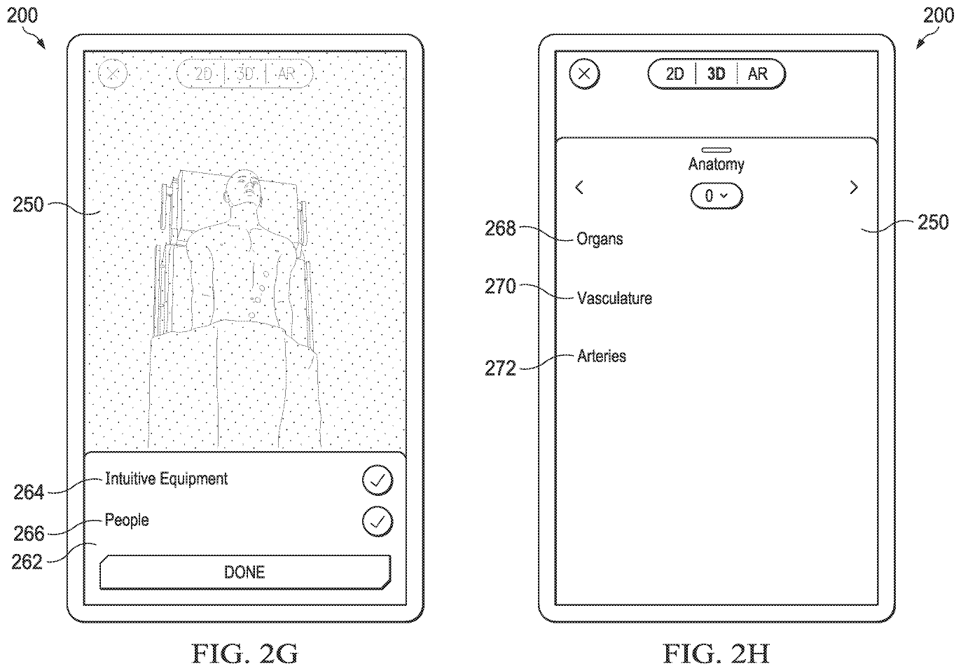

FIG. 2A illustrates a mobile user interface device display 200. The display 200 may include a user interface 204 for receiving a procedure type input 112 indicating a procedure type to be performed with a robot-assisted medical system. The procedures types may be offered for selection from a menu including a menu option 206 and a menu option 208. Any number of menu options representing any number of procedure types may be displayed for selection. In alternative embodiments, the procedure type input 112 may be indicated in other ways such as choosing from a drop-down menu, searching an index, or other know selection techniques. In various embodiments the procedures that may be selected include general surgical procedures including ventral and inguinal hernia repair and bariatric procedures. In various embodiments the procedures that may be selected include colorectal surgical procedures including colectomy and rectal resection. In various embodiments the procedures that may be selected include gynecological surgical procedures including hysterectomy and myomectomy. In various embodiments the procedures that may be selected include urology surgical procedures including prostate, bladder and kidney cancer surgery. In various embodiments the procedures that may be selected include thoracic surgical procedures including lobectomy and mediastinal mass surgery. In various embodiments the procedures that may be selected include cardiac surgical procedures including mitral valve repair. In various embodiments the procedures that may be selected include head and neck surgical procedures including throat cancer procedures. In various embodiments the procedures may include diagnostic or investigative procedures including biopsies.

FIG. 2B illustrates the mobile user interface device display 200 including a user interface 210 for indicating inputs to the selected procedure. The interface 210 may include, for example, a button 212 for receiving input of the surgeon information 114, a button 214 for receiving facility information 116, a button 216 for receiving input of the staff information 118, a button 218 for receiving input of patient information 120, and a button 220 for receiving input of an indication of requested guidance 122. The user interface 210 may include other input mechanisms for entering inputs to the selected procedure. The surgeon information 114 may include, for example, identification information, physical characteristic information (e.g., height, dominant hand), training information, credential information, preference information, history with the staff members, and/or a database of prior procedure information for the surgeon.

The facility information 116 may include, for example, geographic location information for the medical facility where the procedure will be performed, room information, utilities information, available equipment information, or other information about the facility where the selected medical procedure may be performed. In some embodiments, selecting the facility input button 214 may prompt a user to record or capture spatial information. For example, a measurement device for measuring room dimensions using, for example, a rangefinder, lidar system, camera, or other measurement tool that may be a single purpose device or may be incorporated in to the mobile user interface device such as a phone, tablet, or laptop. In some embodiments a camera may capture facility information about a room including, for example, equipment location, utility outlet location, furniture location, and/or door location.

The staff information 118 may include, for example, number of staff members, identification information, physical characteristic information (e.g., height, dominant hand), training information, credential information, preference information, history with the surgeon, and/or a database of prior procedure information for the staff members.

The patient information 120 may include, for example, identification information, gender information, medical history, physical characteristic information (e.g., height, weight), pre-operative medical images (e.g. CT, MRI, X-ray images), information about disease progression, prior medical procedure information, and/or monitored information (e.g. blood pressure, blood oxygenation level, pulse). The patient information may be provided by inputs from, for example, the surgeon, the clinical staff, or a stored database of patient information. In some embodiments, the patient information may include expected or planned patient positioning information, including location and orientation of portions of the patient anatomy including head, torso, and limbs on the operating table. Head rests, pads, supports, and/or other positioning fixtures on the operating table may be used to determine the expected or planned patient positioning. In some embodiments, the patient information may include actual sensed patient positioning information, including location and orientation of portions of the patient anatomy including head, torso, and limbs on the operating table. Cameras, pressure sensors, force sensors, or other sensing systems in or around the operating table may be used to determine patient position during a procedure.

The guidance 122 may include expert recommendations or prior expert actions taken in performing the selected procedure type. The guidance may include an expert's preferred set-up configuration, instrument choice, patient position and orientation, process sequencing, or other suggestions or best practices for performing the selected procedure. The guidance may also or alternatively include a template procedure which may be a generic plan that may be customized based on other inputs 110. The guidance may also or alternatively include a preferred plan (or component steps of a plan) previously implemented by the surgeon, a plan (or component steps of a plan) identified by the surgeon as preferred, or a plan (or component steps of a plan) identified by the surgeon as disfavored. The guidance 122 may be stored in a computer memory for later access, for example, in a subsequent procedure, or may be live guidance information provided from a co-located expert or a remotely located expert.

At the process 102, the inputs 112-124 may be used with reference to or in combination with prior procedure information 124 to generate the procedure plan. The prior procedure information 124 may include information about prior procedures of the same procedure type performed, for example, by the same or a different surgeon, in the same or a different facility, with the same or a different staff, or on the same or a different patient. The prior procedure information 124 may include best practices or practices to avoid based on, for example, efficiency, effectiveness, patient outcome, or surgeon and staff recorded preferences. The prior procedure information 124 may be generated based on evaluations of prior implemented procedures as described below.

The generated procedure plan may include a procedure overview. FIG. 2C illustrates the mobile user interface device display 200 including a user interface 230 for providing an overview of the selected procedure. The procedure overview may include a procedure description 232, a catalog 234 of instruments and supplies to be used during the procedure, a set 236 of procedure instructions, and a trigger 238 to initiate the selected procedure.

After selected procedure is initiated, a plurality of modules of the selected procedure may be presented. FIG. 2D illustrates the mobile user interface device display 200 including a user interface 240 with a selection bar 242 and a selection indicator 244. The selection bar 242 includes references for a plurality of modules (0-6) that correspond to modules or sub-units of the selected procedure. The selection indicator 244 is movable relative to the selection bar 242 to indicate a choice of a selected module. For example, the module 0 may correspond to an anatomy module. The module 1 may correspond to an initial exposure and set-up module. The module 3 may correspond to a vascular control module. The remaining modules may correspond to sub-units of the selected procedure. A procedure may include any number of modules or sub-units. The same type of procedure may even have a different number of models based on the customization provided by the inputs 110.

After the module is selected, a virtual image of the medical environment may be displayed. FIG. 2E illustrates the mobile user interface device display 200 including a user interface 250 for module 0 illustrating an image of a patient anatomy 252 with proposed surgical port placements 253. In the image, the patient anatomy 252 may be positioned on a surgical table 254. In some examples, other equipment such as a robot-assisted manipulator, a surgeon's console, an anesthesia cart, or other components may be included in the image. The images of the patient anatomy 252 and the table 254 may be displayed in a three-dimensional image by selecting a 3D image option 256. In the 3D image, the patient anatomy 252 and the table 254 may be moved with three degrees of rotational freedom. The images of the patient anatomy 252 and the table 254 may be displayed in a two-dimensional image by selecting a 2D image option 258, as shown in FIG. 2F. The images of the patient anatomy 252 and the table 254 may be displayed in an augmented reality image by selecting an AR image option 260. In the augmented reality image, a camera may capture a still or motion image of the medical environment, and the image of the patient anatomy 252 and the table 254 may be superimposed on or otherwise combined with the image of the medical environment to demonstrate how the patient anatomy and the table may be arranged in the actual medical environment space. The patient in the actual medical environment space may be aligned and scaled with the image of the patient anatomy 252 so that the locations of the port placements 253 may be accurately located in the medical environment space. As shown in FIG. 2G, a menu 262 may be presented by selecting an option 263 that allows a user to toggle on and off the images of the equipment with a toggle switch 264 and the images of the people with a toggle switch 266.

As shown in FIG. 2H, the user interface 250 for module 0 may further illustrate organ information 268 describing the anatomic organs that may be involved in the planned procedure, vasculature information 270 describing blood vessels that may be involved in the planned procedure, and arterial information 272 describing arteries that may be involved in the planned procedure.

FIG. 2I illustrates the mobile user interface device display 200 including a user interface 280 for module 1 illustrating instructions for a set-up of a robot-assisted manipulator 282 for performing the planned procedure on the patient anatomy 252. The set-up module 1 may include 2D or 3D images of the recommended orientation of the patient anatomy 252 and the table 254, the recommended port placements 253, the recommended placement of the manipulator 282 relative to the patient anatomy 252 and table 254, docking instructions for docking manipulator 282 to the patient, the recommended and optional instruments, the recommended configuration of other furniture or components in the medical environment, the recommended manipulator set-up joint 284 arrangement, and/or other orientations and placements of objects in the medical environment space. The set-up module 1 may also include a menu to select further explanation of steps 286 in the set-up procedure and videos 288 demonstrating the procedure or steps in the procedure. Each of the modules 0-6 may include 2D images, 3D images, sub-process instructions, instructional videos, kinematic information for the robot-assisted manipulator, descriptions of the impacted anatomy, explanation of instruments used, explanations for the equipment used, and any other text, graphics, videos, or interactive communication tools that may be useful in performing the steps of the module.

Referring again to FIG. 100, at a process 104, performance metrics may be generated during and/or after the implementation of the procedure. As the generated procedure plan is implemented using the robot-assisted manipulator, diversions from the generated plan may occur due to expected and unexpected circumstances such as conditions encountered in the patient anatomy, manipulator performance, staff response to encountered conditions with the patient or manipulator, and/or surgeon response to encountered conditions with the patient or manipulator. During the implementation of the procedure, performance metrics may include kinematic information generated about the robot-assisted manipulator assembly and/or the attached instruments and may include structural information such as the dimensions of the components of the manipulator assembly and/or medical instruments, joint arrangement, component position information, component orientation information, and/or port placements. Kinematic information may also include dynamic kinematic information such as the range of motion of joints in the teleoperational assembly, velocity or acceleration information, and/or resistive forces. The structural or dynamic kinematic constraint information may be generated by sensors in the teleoperational assembly that measure, for example, manipulator arm configuration, medical instrument configuration, joint configuration, component displacement, component velocity, and/or component acceleration. Sensors may include position sensors such as electromagnetic (EM) sensors, shape sensors such as fiber optic sensors, and/or actuator position sensors such as resolvers, encoders, and potentiometers.

Performance metrics may also include elapsed times for the overall procedure, elapsed times for each or a plurality of the discrete sub-units of the procedure, elapsed times to complete activities such as a tool change or a maintenance activity. In some examples, performance quality metrics may include the accuracy of a human response in complying with instructions. For example, if a tool change is indicated for a first manipulator arm, but a staff member instead changes the tool at a second manipulator arm, the performance metric may indicate a non-compliance with instructions. The performance metrics may be binary metrics such as compliance/non-compliance or may be continuous. Continuous metrics may include, for example, a total staff distance travelled, a quantity of mistakes or unplanned incidents during a procedure, a quantity of instruments used, a quantity of instruments damaged, a quantity of tools changed, or a severity metric to describe the types of human interventions required during the implemented procedure.

Performance metrics may also include post-procedure measures including patient outcome quality metrics such as blood loss during and/or after procedure, patient recuperation time, patient readmissions to a medical facility for a related complication, and patient time to discharge, patient post-procedure infection. Post-procedure measures may also include any damage or measures of wear on the manipulator assembly.

During the implementation of the procedure, the performance metrics and/or information about the real-time status of the procedure may be communicated to, for example, the surgeon, the clinical staff, a mentor or supervisor, a facility logistics organization. The metrics and/or status information may be presented in any sensory form including on a display such as the operator interface display, an auxiliary stationary or movable display component, or on a mobile display. The real-time status information may include a listing (e.g. a check list) of procedure steps with an indication of which steps have been performed and which steps remain to be performed. The real-time status information may be useful during staff change-overs or for mentor intervention during an on-going procedure. Other forms of presentation may include auditory information such as an announcement of, for example, an elapsed time or feedback about compliance to the planned procedure.

At a process 106, the implemented procedure may be evaluated based on the performance metrics. The performance metrics may be compared to standards developed by expert surgeons and staff, benchmarks developed by analyzing multiple prior procedures, and or the standards provided by the planned procedure. For example, the kinematic information from the implemented procedure may be compared to the kinematic information recommended by the procedure plan and an evaluation may be made as to whether and as to what extent the implemented kinematic information matched the planned kinematic information. In some examples, the evaluation may generate a score. Evaluation of the performance metrics, including the elapsed times, may provide an indication of where (e.g., in which sub-unit) in the procedure delays or mistakes occurred. In some embodiments, evaluating the implemented procedure may include comparing the performance metric to benchmark metric and identifying a suboptimal result such as a delay or a mistake. In some embodiments, evaluating the implemented procedure may include comparing the performance metric to benchmark metric and identifying a model result that compares favorably to the benchmark metric. In some embodiments, the evaluation of the procedure may be based on actions or performance observed by surgeons, staff, cameras, or other sensors within the environment. In some embodiments, the performance metrics may be objective (e.g., measured data) or at least partially subjective (e.g., human observation based on training and experience).

Optionally, in some embodiments as indicated by the feedback loop between process 106 and process 102, the results of the evaluation may be used during an in-process implementation of the procedure to adjust, modify, update, or otherwise recalculate the subsequent stages of the procedure plan. Thus, the intra-procedure evaluations may allow the procedure plan to be dynamic and continuously responsive to observations, sensor data, and other inputs about the patient and environment during the procedure. For example, during a patient port establishment process, the actual placement of the ports may be different from the placement of the ports recommended by the procedure plan. These revisions may be determined based, for example, on patient anatomy, surgical experience, staff experience, or other considerations that may or may not have been included in the determination of the original procedure plan. These revised port locations may be used to revise the subsequent steps of the procedure plan as the procedure is being implemented.

As shown in FIG. 1, the results of the evaluation at process 106 may be fed to the prior procedure information input 124 to improve the quality of the inputs in the generation of subsequent procedure plans. Both suboptimal results and model results from the evaluation of the implemented procedure may provide useful information for improving subsequent procedures. For example, a second procedure plan based on the stored procedure evaluation information and a second plurality of procedure inputs may be generated at a later implementation of process 102. At an optional process 108, the results of the evaluation may be provided as performance feedback to the surgeon, the staff, or the facility for training and professional growth. In some embodiments, the performance feedback may be delivered during the implementation of the procedure

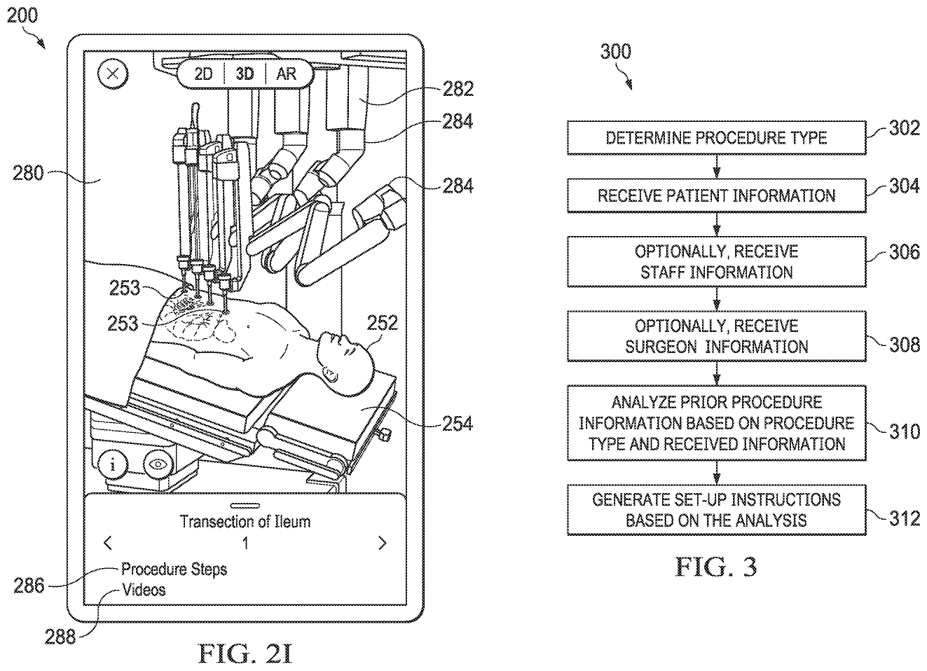

The method 100 of FIG. 1 illustrates a process that may be used for generation of any of a variety of procedure plans that may be used for a full medical procedure such as a colectomy or for a portion of the full procedure where the portions of the full procedure may be, for example, performed by different teams of staff or surgeons. FIG. 3 is a flowchart illustrating a method 300 for generating a portion of a procedure, namely the set-up of the robot-assisted manipulator, peripheral medical components, and the patient at the beginning of a medical procedure. At a process 302, the procedure type to be performed with the robot-assisted medical system may be input at a user interface (e.g., user interface 204). As previously described, the procedures types may be offered for selection from a menu or via other selection techniques. The procedure types may include various surgeries including general, colorectal, gynecological, urology, thoracic, cardiac, and head/neck or may include various diagnostic or investigative procedures. At a process 304 patient information (e.g. patient information 120) may be received. At an optional process 306, staff information (e.g., staff information 118) may be received, and at an optional process 308, surgeon information (e.g. surgeon information 114) for the surgeon operating the robot-assisted manipulator may be received.

At a process 310, prior procedure information may be analyzed or accessed depending on the procedure type and the patient, staff, and/or surgeon information. For example, prior procedure information for the same type of procedure with a surgeon and staff having similar experience levels and a patient having a similarly located target tissue (e.g. a tumor) may yield recommendations for an optimal set-up of the robot-assisted medical system to achieve, for example, the most effective, most efficient, or safest medical procedure. Over time, the analysis, which may include machine learning based on the prior procedure information, may yield customized recommendations for the optimal procedure set-up based on the particular set of inputs. For example, the analysis may include identifying a model prior procedure from the prior procedure information based on common inputs such as the surgeon, the patient characteristics, and/or the level of staff training. In some examples, the analysis may include combining information from multiple prior procedures stored as prior procedure information. In some examples, the analysis may include an analysis of performance metrics including kinematic scores based on kinematic information from a robot-assisted manipulator generated during a prior procedure. In some examples, the analysis may include an analysis of elapsed times for a prior procedure or a sub-unit of a prior procedure. In some examples, the analysis may include analysis of a quality metric such as the quality of staff interventions in the prior procedure or quality of patient outcomes from the prior procedure.

At a process 312, set-up instructions may be generated based on the analysis of the prior procedure information. The set-up instructions may be provided on, for example, a display device 200 and may be used train or instruct the medical staff on an optimal configuration for the robot-assisted system, peripheral components, and the patient in the medical environment. As described above in method 100, the implementation of the set-up plan may be evaluated against the generated set-up plan to provide feedback to the staff regarding reasons mistakes were made during the implementation, reasons for time delays, or reasons for patient outcomes. This feedback may provide personalized training for particular surgeons, types of procedures, and types of patients.

Optionally, the set-up instructions may be displayed on a display device (e.g. display device 200). The displayed set-up instructions may include an augmented reality image including a live image of the medical environment with at least one virtual image of a component (e.g. the manipulator, an instrument, a peripheral component) combined with the live image. In some examples, the displayed set-up instructions may include a video demonstration from a previously recorded procedure.

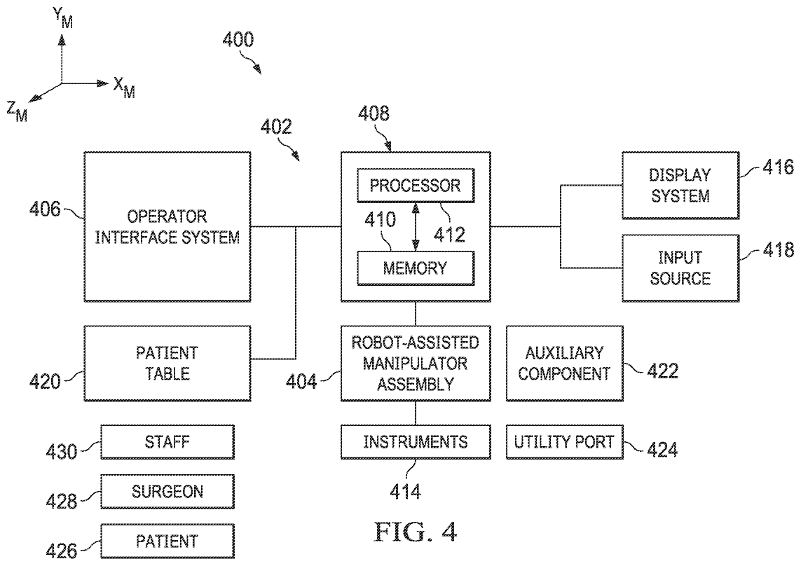

Any of the procedure plans, including set-up plans, described above may be implemented with a robot-assisted medical system in a medical environment. FIG. 4 illustrates one example of a medical environment 400 having a medical environment frame of reference (X_{M}, Y_{M}, Z_{M}) including a robot-assisted medical system 402 that may include components such as a robot-assisted manipulator assembly 404, an operator interface system 406, and a control system 408. In one or more embodiments, the system 402 may be a robot-assisted medical system that is under the teleoperational control of a surgeon. In alternative embodiments, the medical system 402 may be under the partial control of a computer programmed to perform the medical procedure or sub-procedure. In still other alternative embodiments, the medical system 402 may be a fully automated medical system that is under the full control of a computer programmed to perform the medical procedure or sub-procedure with the medical system 402. One example of the medical system 402 that may be used to implement the systems and techniques described in this disclosure is the da Vinci® Surgical System manufactured by Intuitive Surgical Operations, Inc. of Sunnyvale, California. The medical environment 400 may be an operating room, a surgical suite, a medical procedure room, or other environment where medical procedures or medical training occurs.

The control system 408 may include at least one memory 410 and a processing unit including at least one processor 412 for effecting communication, control, and data transfer between components in the medical environment. Any of a wide variety of centralized or distributed data processing architectures may be employed in the control system 408. Similarly, the programmed instructions may be implemented as a number of separate programs or subroutines, or they may be integrated into a number of other aspects of the systems described herein, including teleoperational systems. In one embodiment, the control system 408 may support any of a variety of wired communication protocols or wireless communication protocols such as Bluetooth, IrDA, HomeRF, IEEE 802.11, DECT, and Wireless Telemetry. In some embodiments, the control system 408 may be in a different environment, partially or entirely remote from the manipulator assembly 404 and the operator interface system 406, including a different area of common surgical environment, a different room, or a different building.

The manipulator assembly 404 may be referred to as a patient side cart. One or more medical instruments 414 (also referred to as a tools) may be operably coupled to the manipulator assembly 404. The medical instruments 414 may include end effectors having a single working member such as a scalpel, a blunt blade, a needle, an imaging sensor, an optical fiber, an electrode, etc. Other end effectors may include multiple working members, and examples include forceps, graspers, scissors, clip appliers, staplers, bipolar electrocautery instruments, etc. The number of medical instrument 414 used at one time will generally depend on the medical procedure and the space constraints within the operating room among other factors. A medical instrument 414 may also include an imaging device. The imaging instrument may comprise an endoscopic imaging system using optical imaging technology, or comprise another type of imaging system using other technology (e.g. ultrasonic, fluoroscopic, etc.). The manipulator assembly 404 may include a kinematic structure of one or more links coupled by one or more non-servo controlled joints, and a servo-controlled robotic manipulator. In various implementations, the non-servo controlled joints can be manually positioned or locked, to allow or inhibit relative motion between the links physically coupled to the non-servo controlled joints. The manipulator assembly 404 may include a plurality of motors that drive inputs on the medical instruments 414. These motors may move in response to commands from the control system 408. The motors may include drive systems which when coupled to the medical instrument 414 may advance the medical instrument into a naturally or surgically created anatomical orifice in a patient. Other motorized drive systems may move the distal end of the medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the motors can be used to actuate an articulable end effector of the instrument for grasping tissue in the jaws of a biopsy device or the like. Kinematic information about the manipulator assembly 404 and/or the instruments 414 may include structural information such as the dimensions of the components of the manipulator assembly and/or medical instruments, joint arrangement, component position information, component orientation information, and/or port placements. Kinematic information may also include dynamic kinematic information such as the range of motion of joints in the teleoperational assembly, velocity or acceleration information, and/or resistive forces. The structural or dynamic kinematic constraint information may be generated by sensors in the teleoperational assembly that measure, for example, manipulator arm configuration, medical instrument configuration, joint configuration, component displacement, component velocity, and/or component acceleration. Sensors may include position sensors such as electromagnetic (EM) sensors, shape sensors such as fiber optic sensors, and/or actuator position sensors such as resolvers, encoders, and potentiometers.

The operator interface system 406 allows an operator such as a surgeon or other type of clinician to view images of or representing the procedure site and to control the operation of the medical instruments 414. In some embodiments, the operator interface system 406 may be located in the same room as a patient during a surgical procedure. However, in other embodiments, the operator interface system 406 may be located in a different room or a completely different building from the patient. The operator interface system 406 may generally include one or more control device(s) for controlling the medical instruments 414. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and the like. In some embodiments, the control device(s) will be provided with the same degrees of freedom as the medical tools of the robotic assembly to provide the operator with telepresence; that is, the operator is provided with the perception that the control device(s) are integral with the tools so that the operator has a sense of directly controlling tools as if present at the procedure site. In other embodiments, the control device(s) may have more or fewer degrees of freedom than the associated medical tools and still provide the operator with telepresence. In some embodiments, the control device(s) are manual input devices which move with six degrees of freedom, and which may also include an actuatable handle for actuating medical tools (for example, for closing grasping jaw end effectors, applying an electrical potential to an electrode, capture images, delivering a medicinal treatment, and the like). The manipulator assembly 404 may support and manipulate the medical instrument 414 while an operator views the procedure site through a display on the operator interface system 406. An image of the procedure site can be obtained by the imaging instrument, such as a monoscopic or stereoscopic endoscope, which can be manipulated by the manipulator assembly 404.

Another component that may, optionally, be arranged in the medical environment 400 is a display system 416 that may be communicatively coupled to the control system 408. The display system 416 may display, for example, images, instructions, and data for conducting a robot-assisted procedure. Information presented on the display system 416 may include endoscopic images from within a patient anatomy, guidance information, patient information, and procedure planning information. In some embodiments, the display system may be supported by an electronics cart that allows for mobile positioning of the display system. In some embodiments the display system may be the display device 200. Multiple display systems may be present in the medical environment 400.

An input source 418 may be communicatively coupled to the control system 408 or may be stored in the memory 410. The input source 418 may store one or more of the inputs 110 and/or may be a user interface for receiving one or more of the inputs 110. In some embodiments, the input source 418 may be a database stored outside of the medical environment 400 and accessed by the control system 408. In some embodiments a display system 416 and the input source 418 may be a common device.

Other components in the medical environment 400 that may or may not be communicatively coupled to the control system 408 may include a patient table 420 and an auxiliary component 422 such as an instrument table, an instrument basin, an anesthesia cart, a supply cart, a cabinet, and seating. Other components in the medical environment 400 that may or may not be communicatively coupled to the control system 408 may include may include utility ports 424 such as electrical, water, and pressurized air outlets.

People in or capable of entering the medical environment 400 may include the patient 426 who may be positioned on the patient table 420, a surgeon 428 who may access the operator interface system 406, and staff members 430 which may include, for example surgical staff or maintenance staff.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

Any alterations and further modifications to the described devices, systems, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately.

Various systems and portions of systems have been described in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom).

Although some of the examples described herein refer to surgical procedures or instruments, or medical procedures and medical instruments, the techniques disclosed optionally apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system,” are analogous.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A system comprising:
a processor; and
a memory having computer readable instructions stored thereon, the computer readable instructions, when executed by the processor, cause the system to:
generate a procedure plan for performing a procedure with a robot-assisted manipulator, wherein the procedure plan is based on a first plurality of procedure inputs;
generate a performance metric from the implementation of the procedure;
evaluate the implemented procedure based on the performance metric to generate procedure evaluation information;
store the procedure evaluation information; and
generate a second procedure plan based on the stored procedure evaluation information and a second plurality of procedure inputs.

2. The system of claim 1, wherein the first plurality of procedure inputs includes a procedure type for the procedure.

3. The system of claim 1, wherein the first plurality of procedure inputs includes a surgeon information.

4. The system of claim 1, wherein the first plurality of procedure inputs includes patient information.

5. The system of claim 1, wherein the first plurality of procedure inputs includes prior procedure information.

6. The system of claim 1, further comprising:
displaying the procedure plan on a display device in an environment of the robot-assisted manipulator.

7. The system of claim 6, wherein the displayed procedure plan includes an augmented reality image including a live image of the environment of the robot-assisted manipulator and a virtual image of at least one component in the environment.

8. The system of claim 6, wherein the displayed procedure plan includes a video demonstration of at least a portion of a previously recorded procedure.

9. The system of claim 1, wherein the procedure includes a set of set-up instructions.

10. The system of claim 1, wherein the performance metric is a score based on kinematic information from the robot-assisted manipulator during the implemented procedure.

11. The system of claim 1, wherein the performance metric is an elapsed time for at least one component process of the implemented procedure.

12. The system of claim 1, wherein the performance metric is a quantity of staff interventions during the implemented procedure.

13. The system of claim 1, wherein the performance metric is an outcome quality metric.

14. The system of claim 1, wherein evaluating the implemented procedure includes comparing the performance metric to a benchmark metric and identifying a suboptimal result.

15. The system of claim 14, wherein the suboptimal result is a delay.

16. The system of claim 14, wherein the suboptimal result is a mistake.

17. The system of claim 1, wherein evaluating the implemented procedure includes comparing the performance metric to a benchmark metric and identifying a model result.

18. The system of claim 1, further comprising providing a feedback communication.

19. The system of claim 1, further comprising generating a revised procedure plan after evaluating the implemented procedure.

20-33. (canceled)

34. The system of claim 1, wherein the second procedure plan includes a set of set-up instructions for a second procedure.
